(19) United States
(12) Patent Application Publication
(10) Pub. No.: US 2015/0098867 A1
(43) Pub. Date: Apr. 9, 2015

(54) ODOR SOURCE RELEASE BASED ON ODOR DETECTION AND USER PREFERENCE INFORMATION

(71) Applicant: International Business Machines Corporation, Armonk, NY (US)

(72) Inventors: Alredo Aldereguia, Cary, NC (US); Jeffrey R. Hamilton, Pittsboro, NC (US); Clifton E. Kerr, Durham, NC (US); Grace A. Richter, Raleigh, NC (US)

(73) Assignee: International Business Machines Corporation, Armonk, NY (US)

(21) Appl. No.: 14/047,375

(22) Filed: Oct. 7, 2013

Related U.S. Application Data
(63) Continuation of application No. 14/046,289, filed on Oct. 4, 2013.

(51) Int. Cl. G01N 33/00; A61L 9/01

(52) U.S. Cl. CPC G01N 33/0073; A61L 9/01; G01N 33/0008; G01N 33/004; G01N 33/0054; G01N 33/0044
USPC 422/83

(57) ABSTRACT

Systems and methods for odor source release based on odor detection and user preference information are disclosed. According to an aspect, a method includes receiving user preference information associated with an odor. The method also includes detecting the odor. Further, the method includes releasing one or more odor sources based on the user preference information in response to detecting the odor.

### CROSS-REFERENCE TO RELATED APPLICATIONS

This is a continuation application of copending U.S. patent application Ser. No. 14/046,289, filed Oct. 4, 2013 and titled ODOR SOURCE RELEASE BASED ON ODOR DETECTION AND USER PREFERENCE INFORMATION, the content of which is hereby incorporated by reference in its entirety.

### BACKGROUND

1. Field of the Invention

The present invention relates to odor management, and more specifically, to odor source release based on odor detection and user preference information.

2. Description of Related Art

The sense of smell can vary from person to person based on genetics and other naturally occurring factors that affect the olfactory system. As a result, some people can be more or less sensitive to particular odors as compared to other people. Odors to which people are sensitive may be controlled by various techniques. For example, an unpleasant odor may be masked or treated by dispensing a constant deodorizing chemical volume. In many cases, the dispensed chemical volume may be either insufficient or too much depending on the nearby person. Accordingly, there is a need for improved techniques for controlling unpleasant odors.

### BRIEF SUMMARY

Disclosed herein are systems and methods for odor release based on odor detection and user preference information. According to an aspect, a method includes receiving user preference information associated with an odor. The method also includes detecting the odor. Further, the method includes releasing one or more odor sources based on the user preference information in response to detecting the odor.

According to another aspect, a method includes receiving user preference information of first and second users and associated with one or more odors. The method also includes detecting the one or more odors. Further, the method includes determining release levels of a plurality of odor sources based on the detected one or more odors and the user preference information of the first and second users. The method also includes releasing the odor sources based on the determined release levels in response to detecting the one or more odors.

### BRIEF DESCRIPTION OF THE SEVERAL VIEWS OF THE DRAWINGS

FIG. 1 is a block diagram of an example system for odor source release based on odor detection and user preference information in accordance with embodiments of the present invention;

FIG. 2 is a flowchart of an example method for odor source release in accordance with embodiments of the present invention;

FIG. 3 is a flowchart of another example method for odor source release in accordance with embodiments of the present invention;

FIG. 4 is a timing diagram showing example of odor source release and user input over a period of time in accordance with embodiments of the present invention;

FIG. 5 is a timing diagram showing another example of odor source release and user input over a period of time in accordance with embodiments of the present invention;

FIG. 6 is a timing diagram showing another example of odor source release and user input over a period of time in accordance with embodiments of the present invention; and

FIG. 7 is a flowchart of an example method for odor source release in accordance with embodiments of the present invention.

### DETAILED DESCRIPTION

As described herein, there are various embodiments and aspects of the present invention. Particularly, disclosed herein are systems and methods for odor source release based on odor detection and user preference information.

As referred to herein, the term “gas” may be broadly defined to include pure gases and gas mixtures, including solid and liquid particles entrained therein.

As referred to herein, the term “air” should be broadly construed. It can include a scientific definition of “air” and/or other pure gases and gas mixtures and all solid, liquid and gaseous substances entrained therein.

As referred to herein, the term “odor detector” can be a device that has been calibrated or otherwise has learned through, for example, computer-based learning algorithms and/or programs, how to properly detect and identify a specific target odor. Odor detectors can include, for example, technologies such as ion mobility spectrometry, gas chromatography, mass spectrometry, and liquid chromatography, many or all of which individually or in combination are capable of detecting one or more target odors.

As referred to herein, the term “computing device” should be broadly construed. It can include any type of device capable of presenting a media item to a user. For example, the computing device may be an e-book reader configured to present an e-book to a user. In an example, a computing device may be a mobile device such as, for example, but not limited to, a smart phone, a cell phone, a pager, a personal digital assistant (PDA, e.g., with GPRS NIC), a mobile computer with a smart phone client, or the like. In another example, a computing device can also include any type of conventional computer, for example, a desktop computer or a laptop computer. A typical computing device is a wireless data access-enabled device (e.g., an iPHONE® smart phone, a BLACKBERRY® smart phone, a NEXUS ONE™ smart phone, an iPAD® device, or the like) that is capable of sending and receiving data in a wireless manner using protocols like the Internet Protocol, or IP, and the wireless application protocol, or WAP. This allows users to access information via wireless devices, such as smart phones, mobile phones, pagers, two-way radios, communicators, and the like. Wireless data access is supported by many wireless networks, including, but not limited to, CDPD, CDMA, GSM, PDC, PHS, TDMA, FLEX, ReFLEX, iDEN, TETRA, DECT, DataTAC, Mobitex, EDGE and other 2G, 3G, 4G and LTE technologies, and it operates with many handheld device operating systems, such as PalmOS, EPOC, Windows CE, FLEXOS, OS/9, JavaOS, iOS and Android. Typically, these devices use graphical displays and can access the Internet (or other communications network) on so-called mini- or micro-browsers, which are web browsers with small file sizes that can accommodate the reduced memory constraints of wireless networks. In a representative embodiment, the mobile device is a cellular telephone or smart phone that operates over GPRS (General Packet Radio Services), which is a data technology for GSM networks. In addition to a conventional voice communication, a given mobile device can communicate with another such device via many different types of message transfer techniques, including SMS (short message service), enhanced SMS (EMS), multi-media message (MMS), email WAP, paging, or other known or later-developed wireless data formats. Although many of the examples provided herein are implemented on a mobile device, the examples may similarly be implemented on any suitable computing device, such as a laptop or desktop computer.

As referred to herein, a “user interface” is generally a system by which users interact with a computing device. An interface can include an input for allowing users to manipulate a computing device, and can include an output for allowing the system to present information (e.g., e-book content) and/or data, indicate the effects of the user's manipulation, etc. An example of an interface on a computing device includes a graphical user interface (GUI) that allows users to interact with programs in more ways than typing. A GUI typically can offer display objects, and visual indicators, as opposed to text-based interfaces, typed command labels or text navigation to represent information and actions available to a user. For example, an interface can be a display window or display object, which is selectable by a user of a mobile device for interaction. The display object can be displayed on a display screen of a computing device and can be selected by and interacted with by a user using the interface. In an example, the display of the computing device can be a touch screen, which can display the display icon. The user can depress the area of the display screen at which the display icon is displayed for selecting the display icon. In another example, the user can use any other suitable interface of a computing device, such as a keypad, to select the display icon or display object.

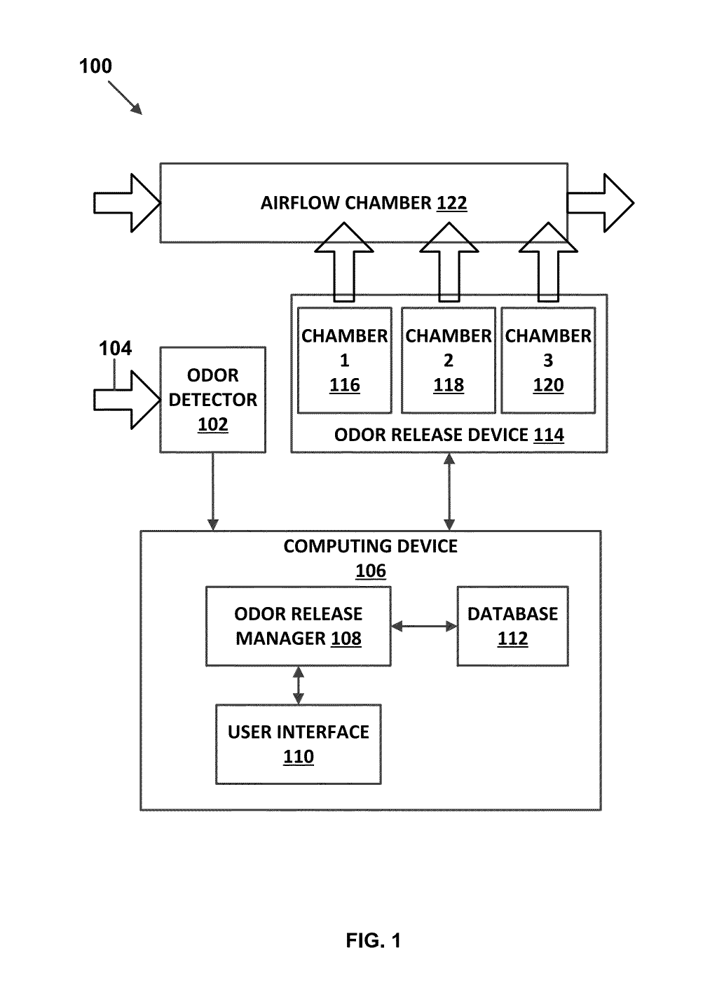

FIG. 1 illustrates a block diagram of an example system 100 for odor source release based on odor detection and user preference information in accordance with embodiments of the present invention. Referring to FIG. 1, the system 100 includes an odor detector 102 configured to detect one or more odors, represented by arrow 104. For example, the odor detector 102 may identify a specific target odor at a very low concentration using a suitable technology. The odor detector 102 may be configured to generate an electrical signal indicative of a presence and/or a concentration(s) of target odor(s) within air. The electrical signal may be output to and received by a computing device 106.

The computing device 106 may include an odor release manager 108, a user interface 110, and a database 112. The computing device 106 may be a desktop computer, a laptop computer, a smartphone, a tablet computer, or the like. Further, the computing device 106 may include one or more suitable input/output (I/O) interfaces (not shown) for electrically communicating with the odor detector 102 and an odor release device 114. The computing device 106 may receive electrical signals from the odor detector 102 that are representative of concentrations of target odors within air surrounding the odor detector 102. Data representative of the detected concentrations of the target odors may be suitably stored in the database 112. Further, the stored data may be timestamped to indicate the detected odor concentrations over time.

The odor release manager 108 may be configured to receive data indicative of the detected odor concentrations. Further, the odor release manager 108 may receive user preference information associated with one or more of the odors. For example, the user preference information may be stored in the database 112 and may indicate a sensitivity of a user to odors. The database 112 may store user preference information for multiple users. A user may enter his or her user preference information by use of the user interface 110. The odor release manager 108 may control the odor release device 114 to release one or more odor sources from within chambers 1 116, 2 118, and/or 3 120. Chamber 116, 118, and 120 may each include a different odor source, such as a deodorizing chemical for neutralizing and/or masking the odor. For example, the odor release manager 108 may generate an electrical signal for controlling the odor release device 114 to open one or more of the chambers 116, 118, and 120. The electrical signal may be communicated to the odor release device 114 for opening one or more of the chambers at varying degrees based on the detected odor 104 and the user preference information. Released odor source may flow into an airflow chamber 122.

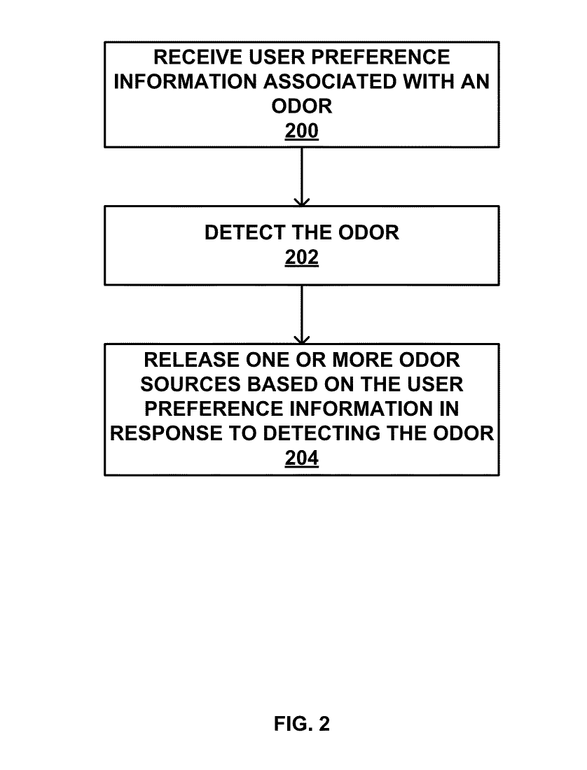

FIG. 2 illustrates a flowchart of an example method for odor source release in accordance with embodiments of the present invention. In this example, the method is described as being implemented by the system 100 shown in FIG. 1, although it should be understood that the method may be implemented by any suitable system. More particularly, the example method is described as being implemented by odor release manager 108 in coordination with other components shown in FIG. 1. The odor release manager 108 may be implemented by hardware, software, firmware, or combinations thereof. For example, the odor release manager 108 may be implemented by one or more processors and memory suitably configured with instructions for implementing the functions described herein.

Referring to FIG. 2, the method includes receiving 200 user preference information associated with an odor. For example, the user may interact with the user interface 110 for inputting a user input indicating sensitivity to one or more odors. The user interface 110 may include a keyboard, mouse, touchscreen display, or any other suitable user interface configured to receive user input. In an example, the user interface 110 may present (e.g., display) information indicating one or more odors. The user may interact with the user interface 110 to select one or more of the odors and the user's sensitivity level to the odor(s). Subsequently, the user input may be received by the odor release manager 108. The user input may be stored in the database 112. Further, for example, the user may be associated with a suitable identifier stored in the database 112. The user identifier may be associated with the selected odor(s) and/or the sensitivity level information.

The method of FIG. 2 includes detecting 202 the odor. For example, the odor detector 102 may detect the odor(s) 104 and communicate to the computing device 106 an electrical signal carrying data indicative of a presence and/or concentration of the odor within the air. The odor detector 102 may be configured to, for example, measure a density of components of the odor(s) 104 within a gas. As an example, the odor types may include, but are not limited to, carbon monoxide, sulfur, hydrogen sulfide, ammonia, and the like. The odor release manager 108 may receive the data indicative of the presence and/or concentration of the odor. The stored data may include information indicating the type of odor(s) and a level (e.g., density or amount) of the odor. Further, the data may be stored in the database 112.

The method of FIG. 2 includes releasing 204 one or more odor sources based on the user presence information in response to detecting the odor. Continuing the aforementioned example, the odor release manager 108 may control the odor release device 114 to release one or more odor sources from within chambers 116, 118, and 120. In an example, the odor source(s) may be released based on the user's sensitivity to the detected odor(s) 104. For example, the odor release manager 108 may lookup the user's sensitivity level information for a detected odor. The odor release manager 108 may control the device to open one or more of the chambers 116, 118, and 120 based on the user's sensitivity level information for the detected odor(s). The odor source that is released may be an odor source that can neutralize and/or mask the detector odor(s). Further, for example, the amount of odor source released may be varied based on the user's sensitivity level. For example, the more a user is sensitive to the odor, the greater the amount of odor source released. The odor release manager 108 may control release of the odor source(s) in response to detection of the odor(s) 104.

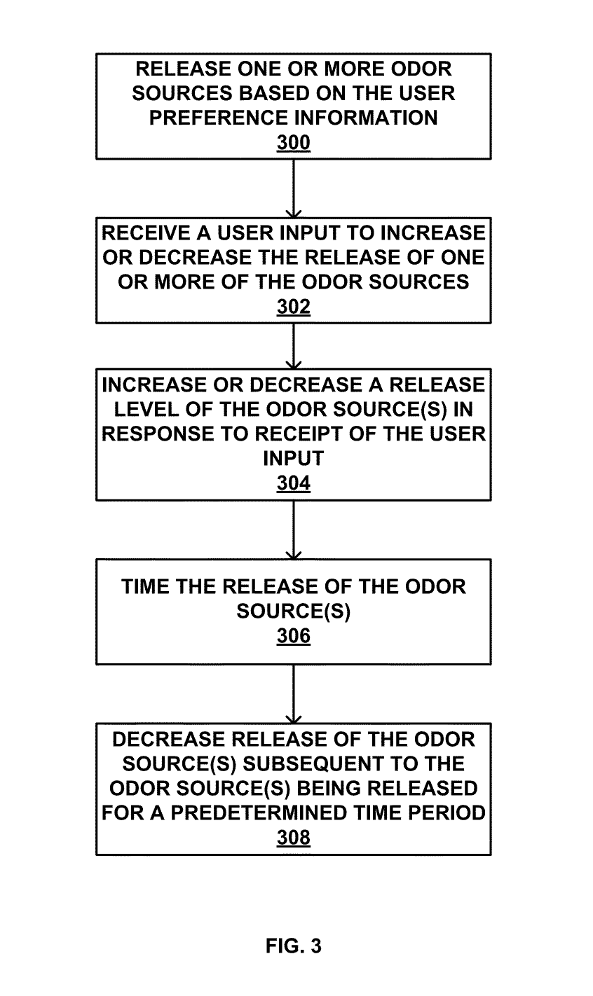

FIG. 3 illustrates a flowchart of another example method for odor source release in accordance with embodiments of the present invention. In this example, the method is described as being implemented by the system 100 shown in FIG. 1, although it should be understood that the method may be implemented by any suitable system. More particularly, the example method is described as being implemented by odor release manager 108 in coordination with other components shown in FIG. 1. The odor release manager 108 may be implemented by hardware, software, firmware, or combinations thereof. For example, the odor release manager 108 may be implemented by one or more processors and memory suitably configured with instructions for implementing the functions described herein.

Referring to FIG. 3, the method includes releasing 300 one or more odor sources based on user preference information. For example, the odor release device 114 may be controlled by the odor release manager 108 to open one or more of the chambers 116, 118, and 120 for releasing the odor sources contained therein. Multiple different odor sources in two or more of the chambers may each be released at a predetermined release level based on the detected odor and/or the user preference information. The odor sources may be released at levels or amounts based on a sensitivity of one or more users to the detected odor(s) 104.

The method of FIG. 3 includes receiving 302 a user input to increase or decrease the release of one or more of the odor sources. Continuing the aforementioned example, the user may interact with the user interface 110 to select an input for either increasing or decreasing the release of odor source(s). For example, the user may touch an icon of a touchscreen display to selectively increase or decrease a level or amount of odor source release. The user input may be received by the odor release manager 108.

The method of FIG. 3 includes increasing or decreasing 304 a release level of one or more odor sources in response to receipt of the user input. Continuing the aforementioned example, the odor release manager 108 may determine a control input for the odor release device 114 for increasing or decreasing the release levels of the odor sources in chambers 116, 118, and/or 120 based on the user input. The odor release manager 108 may subsequently communicate the control input to the odor release device 114 to increase or decrease the release level(s) of the odor source(s).

The method of FIG. 3 includes timing 306 the release of the one or more odor sources. Continuing the aforementioned example, the odor release manager 108 may be configured to time the release of the odor source(s). For example, the odor release manager 108 may initiate a timer when an instruction is communicated to the odor release device 114 to release an odor source. The timing information may be stored in the database 112.

The method of FIG. 3 includes decreasing 308 release of the odor source(s) subsequent to the odor source(s) being released for a predetermined time period. For example, the odor release manager 108 may control the odor release device 114 to decrease release of an odor source at a time after the odor release device 114 was instructed to release the odor source. In this way, odor source release can be automatically reduced after a predetermined time period has passed since the initial release of the odor source.

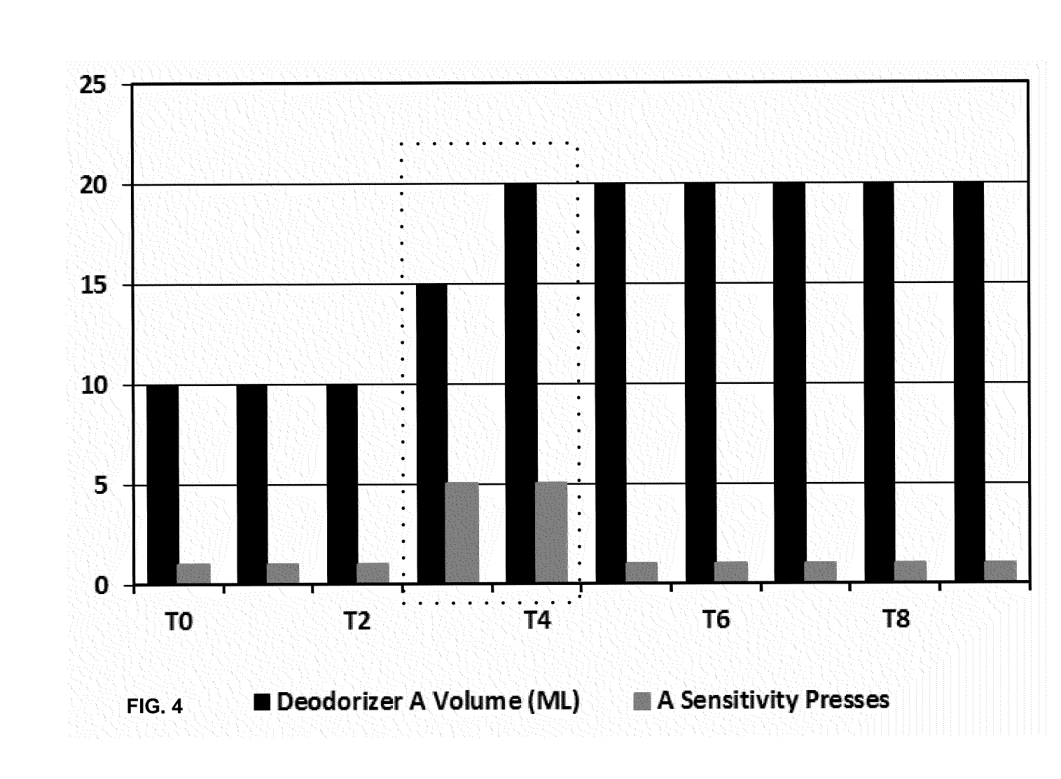

FIG. 4 illustrates a timing diagram showing example of odor source release and user input over a period of time in accordance with embodiments of the present invention. Referring to FIG. 4, the odor source release and user input are shown at times T0-T9. At times T0-T2, the odor source release and user input are the same. At times T3 and T4, the user inputs 2 commands to indicate a sensitivity of the user to an odor for increasing the odor source release as shown at times T3-T9. Particularly, the output level is increased by 5 mL each time the user selects the input.

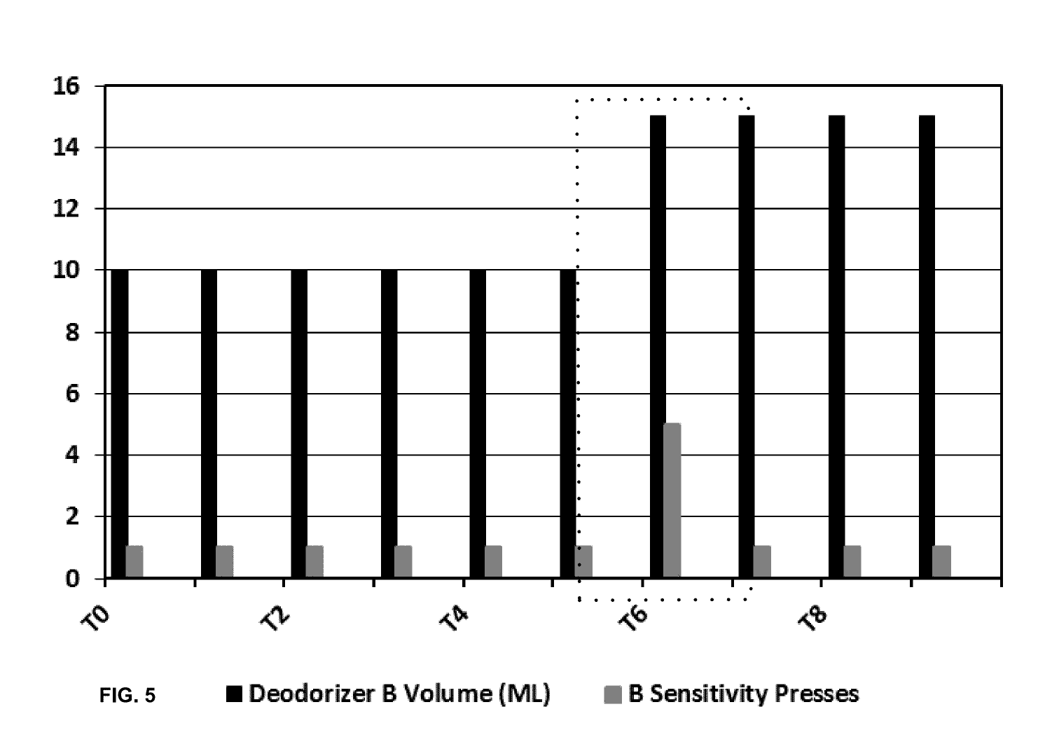

FIG. 5 illustrates a timing diagram showing another example of odor source release and user input over a period of time in accordance with embodiments of the present invention. Referring to FIG. 5, the odor source release and user input are shown at times T0-T9. At times T0-T6, the odor source release and user input are the same. At time T7, the user inputs a single command to indicate a sensitivity of the user to an odor for increasing the odor source release as shown at times T7-T9. Particularly, the output level is increased by a factor of 1.5 on the user input. Thus, the odor source release increases from 10 mL to 15 mL.

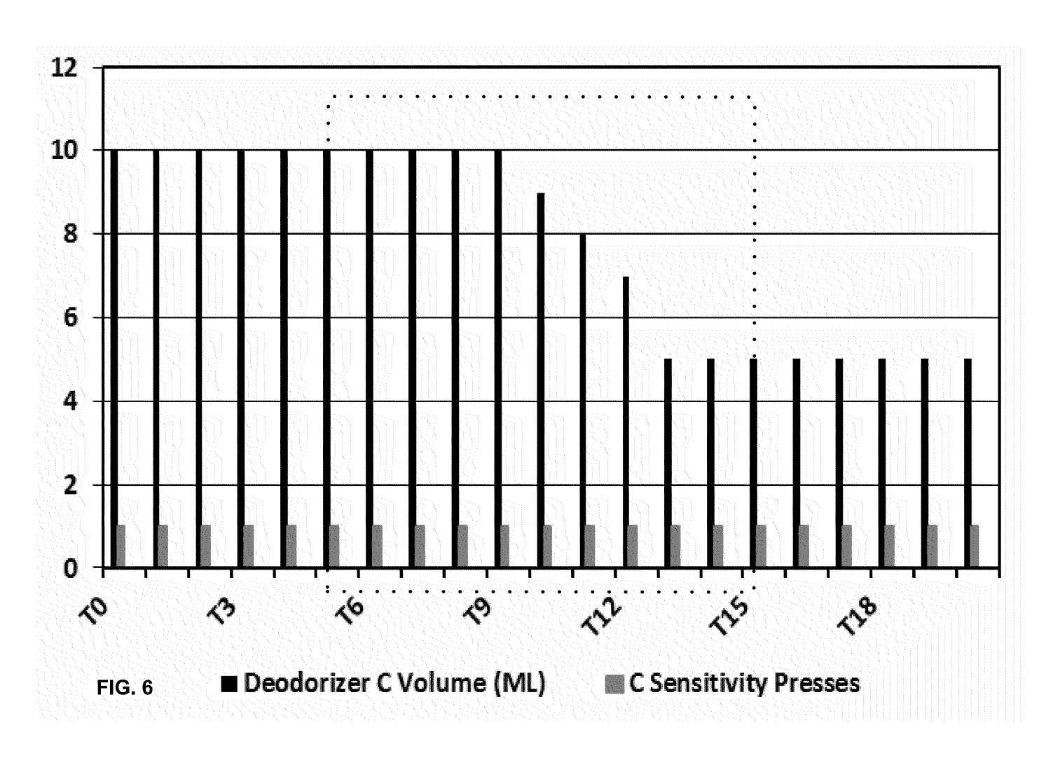

FIG. 6 illustrates a timing diagram showing another example of odor source release and user input over a period of time in accordance with embodiments of the present invention. Referring to FIG. 6, the odor source release and user input are shown at times T0-T21. At times T0-T21, the user input is the same. Beginning at time T10, the odor source release level reduces from 10 mL to 5 mL. Triggering for reduction of the release level may be based upon the user not entering a sensitivity input over a predetermined time period. After expiration of a time period with no user input, the odor source release levels may be reduced as shown, for example.

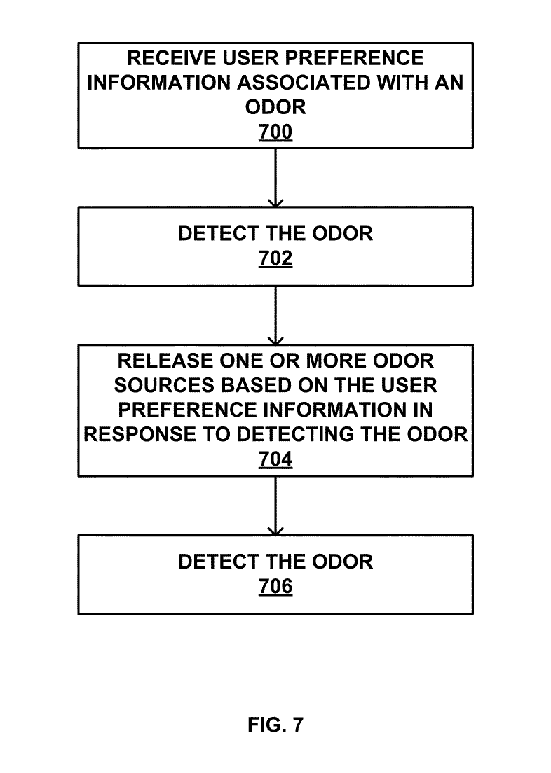

FIG. 7 illustrates a flowchart of an example method for odor source release in accordance with embodiments of the present invention. In this example, the method is described as being implemented by the system 100 shown in FIG. 1, although it should be understood that the method may be implemented by any suitable system. More particularly, the example method is described as being implemented by odor release manager 108 in coordination with other components shown in FIG. 1. The odor release manager 108 may be implemented by hardware, software, firmware, or combinations thereof. For example, the odor release manager 108 may be implemented by one or more processors and memory suitably configured with instructions for implementing the functions described herein.

Referring to FIG. 7, the method includes receiving 700 user preference information of first and second users and associated with one or more odors. For example, 2 users may enter their user preference information into the user interface 110. The user preference information may indicate a sensitivity of each of the users to one or more odors. Further, the method of FIG. 7 includes detecting 702 the odor(s).

In accordance with embodiments of the present invention, the odor detector 102 may detect one or more odors 104. The user interface 110 may indicate a multiple odor types. Multiple users may enter their respective user preference information for indicating sensitivity to the odor types.

The method of FIG. 7 includes determining 704 release levels of multiple odor sources based on the detected odor(s) and the user preference information of the first and second users. Continuing the aforementioned example, the odor release manager 108 may determine release levels for the odor sources within chambers 116, 118, and 118. This determination may be based on the different users' sensitivity to odor types.

The method of FIG. 7 includes releasing 706 the odor sources based on the determined released levels in response to detecting the odor(s). Continuing the aforementioned example, the odor release manager 108 may control the odor release device 114 to release the odor sources in the chambers 116, 118, and 120 in accordance with the determined release levels. Further, for example, the release levels may be controlled based on further inputs by the users for increasing or decreasing the release levels.

In accordance with embodiments of the present invention, the odor release manager 108 may learn preferences to a user and generate a user profile of the user. The user profile may be stored in the database 112. For example, the odor release manager 108 may store user control of the odor release levels in response to detection of odor(s). This may be considered a feedback of user response to odors. In this way, the odor release manager 108 can learn a user's preference and adjust odor release levels in accordance with those preferences.

In accordance with embodiments, the odor release manager 108 may receive odor measurements from the odor detector 102. The odor detector 102 may evaluate the odor type, amount of odor, and stored user sensitivity levels to determine a volume of deodorizing chemicals that may be needed for dispense by the odor release device 114. Calculation of the deodorizing chemical volume may be based on the following formula:

DoV=(Oca*Ds)*S

wherein DoV represents deodorant source output volume, Oca represents a percentage (%) of odorous chemical in the air, Ds represents a standard deodorizer constant (this may vary depending on the type of odor), and S represents a user sensitivity multiplier.

The value of S may be variable and may be dependent on the number of times a user provides input to the output control processor that they are sensitive to the odor. S may be generic and apply to all odor types, or be associated with specific odors, whereby individual sensitivity records can be maintained and adjusted by the user for varying types of odors. These types of odors may be presented to the user in terms such as, but not limited to, “musky,” “putrid” (e.g., egg), “pungent” (e.g., vinegar), floral, and the like. When a user detects an unpleasant odor of such types, the user may provide input to the user interface 110. A feedback from the output environment to the odor detector 102 may be implemented if the odor of deodorizing chemicals starts to irritate the user. Referring to FIGS. 4-6, the diagrams show how the amount of deodorizer volume output into the airflow chamber 122. Particularly, FIGS. 4-6 are associated with the release of different odor sources for masking different respective odors. For example, the different odor sources released in the examples of FIGS. 4-6 may mask “musky,” “putrid,” and “pungent” odors, respectively.

In an example scenario, there may be a number of user sensitivity multipliers (e.g., S1, S2, S3, etc.) based on the number of users in the output environment. For example, the primary user, Jane, may be sensitive to pungent odors, but not musky odors. Jane may provide feedback to the odor release manager 108 about this via the user interface 110. Another user, Joe, may enter the environment and notice a musky odor that Jane does not care about. Joe may interact with the user interface to indicate his sensitivity to the musky odor. In response, an odor source may be released to mask or eliminate the musky odor.

It is noted that systems disclosed herein may be deployed in any suitable environment. For example, the system 100 may be used in a vehicle (e.g., automobile), rooms in a building, or a single room.

As will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium (including, but not limited to, non-transitory computer readable storage media). A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter situation scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The flowchart and block diagrams in the figures illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It should also be noted, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The terminology used herein is for the purpose of describing particular embodiments only and is not intended to be limiting of the invention. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A system comprising:
at least a processor and memory; and
an odor release manager configured to:
receive user preference information associated with an odor; and
detect the odor; and
control an odor release device to release one or more odor sources based on the user preference information in response to detecting the odor.

2. The system of claim 1, further comprising a user interface configured to receive user input indicating sensitivity to the odor.

3. The system of claim 2, wherein the odor release manager is configured to control the odor release device to release one or more odor sources based on the sensitivity.

4. The system of claim 1, wherein the odor release manager is configured to:
receive a user input to increase the release of the one or more odor sources; and
control the odor release device to increase a release level of the one or more odor sources in response to receipt of the user input.

5. The system of claim 1, wherein the odor release manager is configured to:
receive a user input to decrease the release of the one or more odor sources; and
control the odor release device to decrease a release level of the one or more odor sources in response to receipt of the user input.

6. The system of claim 1, wherein the odor release manager is configured to measure a density of components of an odor within a gas.

7. The system of claim 1, wherein the odor release manager is configured to detect one of carbon monoxide, sulfur, hydrogen sulfide, and ammonia.

8. The system of claim 1, wherein the odor release manager is configured to control the odor release device to release a plurality of different odor sources each in a predetermined release level based on the detected odor and the user preference information.

9. The system of claim 1, wherein the odor release manager is configured to:
determine a release level of each of the one or more odor sources based on the user preference information; and
control the odor release device to release the one or more odor sources based on the determined release level.

10. The system of claim 1, wherein the odor release manager is configured to:
time the release of the one or more odor sources; and
control the odor release device to decrease release of the one or more odor sources subsequent to the one or more odor sources being released for a predetermined time period.

11. The system of claim 1, wherein the odor release manager is configured to:
present a user interface that indicates a plurality of odor types;
receive user input indicating sensitivity to one or more of the odor types; and
control the odor release device to release the one or more odor sources based on the user input.

12. The system of claim 11, wherein the one or more odor types comprises multiple odor sources, and
wherein the odor release manager is configured to:
receive user input comprises receiving an indication of a sensitivity level to each of the odor types; and
control the odor release device to release different amounts of odor sources based on the indicated sensitivity levels.

13. A system comprising:
at least a processor and memory; and
an odor release manager configured to:
receive user preference information of first and second users and associated with one or more odors;
detect the one or more odors;
determine release levels of a plurality of odor sources based on the detected one or more odors and the user preference information of the first and second users; and
control an odor release device to release the odor sources based on the determined release levels in response to detecting the one or more odors.

14. The system of claim 13, wherein the odor release manager is configured to receive user input that indicates a sensitivity of each of the first and second users to the one or more odors.

15. The system of claim 13, wherein the odor release manager is configured to:
receive a user input associated with one of the first and second users to increase the release of one or more of the odor sources; and
control the odor release device to increase a release level of the one or more of the odor sources in response to receipt of the user input.

16. The system of claim 13, wherein the odor release manager is configured to:
receive a user input associated with one of the first and second users to decrease the release of one or more of the odor sources; and
control the odor release device to decrease a release level of the one or more odor sources in response to receipt of the user input.

17. The system of claim 13, wherein the odor release manager is configured to measure a density of components an odor within a gas.

18. The system of claim 13, wherein the odor release manager is configured to detect one of carbon monoxide, sulfur, hydrogen sulfide, and ammonia.

19. The system of claim 13, wherein the odor release manager is configured to control the odor release device to release a plurality of different odor sources each in a predetermined release level based on the detected one or more odors and the user preference information.

20. The system of claim 1, wherein the odor release manager is configured to:
present a user interface that indicates a plurality of odor types;
receive user input from the first and second users for indicating sensitivity to the odor types; and
control the odor release device to release the odor sources based on the user input.
